(19) United States
(12) Patent Application Publication
(10) Pub. No.: US 2022/0157185 A1
(43) Pub. Date: May 19, 2022

(54) SYSTEM AND METHOD OF ASSISTING COGNITIVE ABILITY DEVELOPMENT FOR VISUALLY IMPAIRED OR CHILDREN

(71) Applicant: PCT(Power Contents Technology) Co., Ltd., Seongnam-si (KR)

(72) Inventors: Jungil Jung, Yongin-si (KR); Dongkyu Kim, Daegu (KR)

(21) Appl. No.: 17/477,601

(22) Filed: Sep. 17, 2021

(30) Foreign Application Priority Data
Nov. 16, 2020 (KR) 10-2020-0152991

(51) Int. Cl. G09B 5/04; G10L 13/047; H04W 76/14; H04B 5/00; G10L 13/033; G06K 19/07; G06K 7/10

(52) U.S. Cl. CPC G09B 5/04; G10L 13/047; H04W 76/14; G06K 7/10297; G10L 13/033; G06K 19/0723; H04B 5/0031

(57) ABSTRACT

The present invention relates to a system and a method to assist cognitive ability development for the visually impaired or children, and more particularly, to a system and a method of assisting cognitive ability development for the visually impaired or children in which contents of an NFC tag is inputted through a user terminal, the tag is attached to teaching aids, toys, objects, books and the like and an auxiliary engineering appliance is tagged, thereby voice-supporting the content of the tag, so as to assist cognitive ability development of the visually impaired or a child.

### BACKGROUND OF THE INVENTION

### 1. Field of the Invention

The present invention relates to a system and a method to assist cognitive ability development for the visually impaired or children, and more particularly, to a system and a method of assisting cognitive ability development for the visually impaired or children in which contents of an NFC tag is inputted through a user terminal, the tag is attached to teaching aids, toys, objects, books and the like and an auxiliary engineering appliance is tagged, thereby voice-supporting the content of the tag, so as to assist cognitive ability development of the visually impaired or a child.

### 2. Description of the Related Art

In order to develop the cognitive abilities of children with visual impairments, more delicate directions such as learning hearing ability through a voice guide of a guardian or educator are required. In the independent environment without the guardian, learning can be performed only when the child remembers all contents that the child has learned. Although various self-motivating teaching aids for non-impaired children have been developed to enable the children to learn cognitive abilities, teaching aids for self-motivating the learning of the visually impaired children are extremely rare. In addition, although education recently has been actively conducted through cyber lectures, remote classes, and the like, the above education through non-contact classes is merely the superficial class that provides only educational contents such as Braille or sign language to the visually impaired children, and finally, the education for the children maybe burdened on parents. This causes a major factor of stress not only for the impaired or non-impaired children who receives the above education but also for the parents.

Accordingly, the domestic company VOICEYE has developed a technology of supplying ‘voice code’ for voice conversion using QR code in order to support a voice to the visually impaired. However, the QR code is required to be accurately recognized using a smartphone, thereby causing inconvenience for the visually impaired. In addition, in order to educate children, it is very difficult for parents to directly generate QR codes suitable for each teaching aid or book. In addition, the company SAYPEN has developed ‘Saypen’ that reads books for children. However, because the Saypen is used only for textbooks to which the Saypen is applied, it is difficult to use the Saypen in other textbooks having contents other than the contents which are provided by the company SAYPEN. In addition, the Saypen is only applied to books for general non-impaired children, and cannot be used by the visually impaired children using Braille books at all.

Meanwhile, as communication technology develops, the short-distance wireless communication is being used in various fields. As the spread of smartphones is active, a near field communication (NFC) technology among the short-distance wireless communication technologies is being used for electronic payment system, chip for access permission, advertisement for subway and bus, and the like. The NFC communication function can be easily used in smartphones that are currently being distributed, and various functions can be implemented by simply touching an NFC tag, card, sticker or the like.

in order to solve the above problem, technology development has been required, in which the NFC technology is used so that a guardian or educator may directly input various contents without being limited to some contents of an object or book into the NFC tag and configure the contents. In addition, a child may tag an auxiliary appliance on the object or book to which the above NFC tag is attached, so as to primarily recognize the contents the object or the Braille book through a tactile sense, and secondary recognize the contents through a voice, thereby assisting cognitive ability development through tactile and hearing senses.

### SUMMARY OF THE INVENTION

The present invention provides a system and a method to assist cognitive ability development for the visually impaired or children, in which contents of an NFC tag is inputted through a user terminal, the tag is attached to teaching aids, toys, objects, books and the like and an auxiliary engineering appliance is tagged, thereby voice-supporting the content of the tag, so as to assist cognitive ability development of the visually impaired or child.

In order to solve the above problem, the present invention provides a system of assisting cognitive ability development for the visually impaired or children, which includes: an NFC tag module including an antenna and an IC chip and configured to be readable and writable; and an auxiliary engineering appliance, wherein the auxiliary engineering appliance includes: a battery module; an NFC reader module for receiving text information recorded in the NFC tag module by using an NFC communication scheme; a control module for converting the text information received from the NFC reader module into voice information; a speaker for outputting the voice information converted by the control module; and a connector board for connecting the control module to the NFC reader module.

In one embodiment of the present invention, the NFC tag module may include an IC chip in which text information inputted by a user using a dedicated application on a smartphone terminal of the user is recorded.

In one embodiment of the present invention, the NFC reader module may be disposed on one side of the connector board, and the control module, the speaker, and the battery module may be disposed on the other side of the connector board.

In one embodiment of the present invention, the auxiliary engineering appliance may further include a switch module including: a power switch capable of controlling On/Off of the auxiliary engineering appliance; an NFC tagging switch capable of activating tagging of the NFC reader module and the NFC tag module; a Bluetooth pairing switch capable of performing Bluetooth pairing with a smartphone terminal; and a voice control switch capable of controlling speed and volume of a voice outputted to the speaker.

In one embodiment of the present invention, the NFC tag module may be attached to one side of a book, and the NFC tag module may include: a first NFC tag including an antenna and an IC chip; a first adhesive layer disposed on the first NFC tag and having a sectional area corresponding to the first NFC tag; an electromagnetic wave blocking layer disposed on the first adhesive layer and having an area greater than the first NFC tag; a second adhesive layer disposed on the electromagnetic wave blocking layer and having a sectional area corresponding to the electromagnetic wave blocking layer; and a second NFC tag disposed on the second adhesive layer and having an area smaller than the second adhesive layer.

In order to solve the above problem, one embodiment of the present invention provide a method of assisting cognitive ability development by using a system of assisting cognitive ability development for the visually impaired or children. The system of assisting cognitive ability development includes: an NFC tag module including an antenna and an IC chip and configured to be readable and writable; and an auxiliary engineering appliance, wherein the auxiliary engineering appliance includes: a battery module; an NFC reader module for receiving text information recorded in the NFC tag module by using an NFC communication scheme; a control module for converting the text information received from the NFC reader module into voice information; a speaker for outputting the voice information converted by the control module; and a connector board for connecting the control module to the speaker and the NFC reader module, and the method of assisting cognitive ability development includes: inputting, by a user, text information by using a dedicated application on a smartphone terminal of the user, and recording the inputted text information in the NFC tag module by using NFC communication between the NFC tag module and the smart phone terminal; receiving, by the auxiliary engineering appliance, the text information recorded in the NFC tag module by using an NFC communication scheme; converting, by the auxiliary engineering appliance, the text information received from the NFC reader module into voice information; and outputting, by the auxiliary engineering appliance, the voice information converted by the control module.

According to one embodiment of the present invention, the auxiliary engineering appliance for voice-supporting the contents of the NFC tag is used, so that the cognitive ability development of visually impaired or non-impaired children can be improved.

According to one embodiment of the present invention, a guardian or educator can input the contents of the NFC tag through a smartphone terminal of the guardian or educator, so as to directly produce educational materials containing the contents of educational teaching aids and books for children that the guardian or educator has already possessed.

According to one embodiment of the present invention, the system of assisting cognitive ability development can enable the visually impaired or non-impaired children to perform self-motivated learning even in an independent environment without a guardian.

According to one embodiment of the present invention, the user-dedicated application for guardians and educators may be used so that text information to be inputted to the NFC tag module can be more conveniently customized.

According to one embodiment of the present invention, the speed of the voice of the text information inputted into the NFC tag module can be adjusted according to the input of a child using the auxiliary engineering appliance, so that the user-customized voice support can be provided.

According to one embodiment of the present invention, the single-sided NFC tag module has the thickness equal to or less than the height of a braille book, so that the thickness of the braille book can be prevented from being thick even when the NFC tag module is attached to the book.

According to one embodiment of the present invention, the both-sided NFC tag module may be used text information can be inputted to read the contents of both sides of the book even when the NFC module is attached to only one side of the book, so that interference of NFC signals due to attaching a plurality of NFC tag modules can be prevented.

### BRIEF DESCRIPTION OF THE DRAWINGS

FIG. 1 schematically shows the overall configuration of a system to assist cognitive ability development for the visually impaired or child according to one embodiment of the present invention.

FIG. 2 is a diagram for explaining a method of using the cognitive ability development assistance system according to one embodiment of the present invention.

FIG. 3 schematically shows the internal configuration of the auxiliary engineering appliance according to one embodiment of the present invention.

FIG. 4 schematically shows a side view of the auxiliary engineering appliance according to one embodiment of the present invention.

FIG. 5 schematically shows a perspective view and a bottom view of the auxiliary engineering appliance according to one embodiment of the present invention.

FIGS. 6A and 6B schematically show the configuration of the auxiliary engineering appliance according to one embodiment of the present invention.

FIG. 7 is a view for explaining some embodiments that use the system for assisting cognitive ability development according to the embodiment of the present invention.

FIGS. 8A,8B,8C and 8D schematically show an NFC tag module according to one embodiment of the present invention.

FIG. 9 is a diagram for explaining a method of using the cognitive ability development assistance system according to one embodiment of the present invention.

### DETAILED DESCRIPTION OF THE INVENTION

Hereinafter, embodiments of the present invention will be described in detail with reference to the accompanying drawings, so that a person having ordinary skill in the art may easily carry out the present invention. However, the invention may be embodied in various different forms and is not limited to the embodiments described herein. In addition, parts irrelevant to the description are omitted in the drawings to clearly describe the present invention, and like reference numerals designate like parts throughout the specification.

Throughout the specification, when a part is “connected” the other part, the above expression includes not only “directly connected” but also “electrically connected” in which another element is interposed therebetween. In addition, when a part “includes” a certain component, the above expression does not exclude other components, but may further include the other components, unless particularly stated otherwise

In addition, the terms including an ordinal number such as first and second may be used to describe various components, however, the components are not limited by the terms. The terms are used only for the purpose of distinguishing one component from another component. For example, the first component may be referred to as the second component without departing from the scope of the present invention, and similarly, the second component may also be referred to as the first component. The term “and/or” includes any one of a plurality of related listed items or a combination thereof.

In the present specification, the term ‘unit’ includes a unit implemented by hardware, a unit implemented by software, and a unit implemented by both of the hardware and the software. In addition, one unit may be implemented using at least two pieces of hardware, and at least two units may be implemented by one piece of hardware. Meanwhile, “unit” may not be limited to software or hardware, may be configured to be disposed in an addressable storage medium, and may be configured to reproduce at least one processor. Accordingly, as an example, the ‘unit’ includes components such as software components, object-oriented software components, class components, and task components, processes, functions, attributes, procedures, subroutines, segments of program code, drivers, firmware, microcode, circuits, data, databases, data structures, tables, arrays and variables

The function provided within the components and the ‘unit’s may be combined into a smaller number of components and ‘unit’s or further separated into additional components and the ‘unit’s. In addition, the components and ‘unit’s may be implemented to reproduce at least one CPU in a device or a secure multimedia card.

The “user terminal” referred to below may be implemented as a computing device or portable terminal having at least one processor and at least one memory and capable of accessing a server or another terminal in a wired or wireless manner. The computing device, for example, may include a notebook computer installed with a web browser, a smart phone, a tablet, a personal computer (hereinafter referred to as “PC”), a mobile phone, a video phone, an e-book reader, a desktop PC, a laptop PC, a netbook PC, a personal digital assistant (hereinafter referred to as “PDA”), a portable multimedia player (hereinafter referred to as “PMP”), an mp3 player, a mobile medical device, a camera, and a wearable device such as a head-mounted device (hereinafter referred to as “HMD”), an electronic garment, an electronic bracelet, an electronic necklace, an electronic appcessory, an electronic tattoo, or a smart watch.

The portable terminal refers to a wireless communication device that guarantees, for example, portability and mobility, may include all kinds of handheld-based wireless communication device such as personal communication system (PCS), global system for mobile communications (GSM), personal digital cellular (PDC), personal handy phone system (PHS), personal digital assistant (PDA), international mobile telecommunication (IMT)-2000, code division multiple access (CDMA)-2000, W-code division multiple access (W-CDMA), wireless broadband internet (Wibro) terminal.

In addition, the “network” may be implemented by a wired network such as a local area network (LAN), a wide area network (WAN) or a value added network (VAN), and all types of wireless network such as a mobile radio communication network, a satellite communication network, WiFi, 3G, LTE, Bluetooth, Beacon, NFC, or Zigbee.

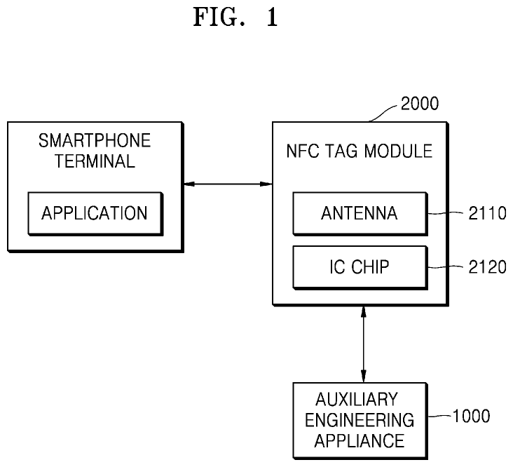

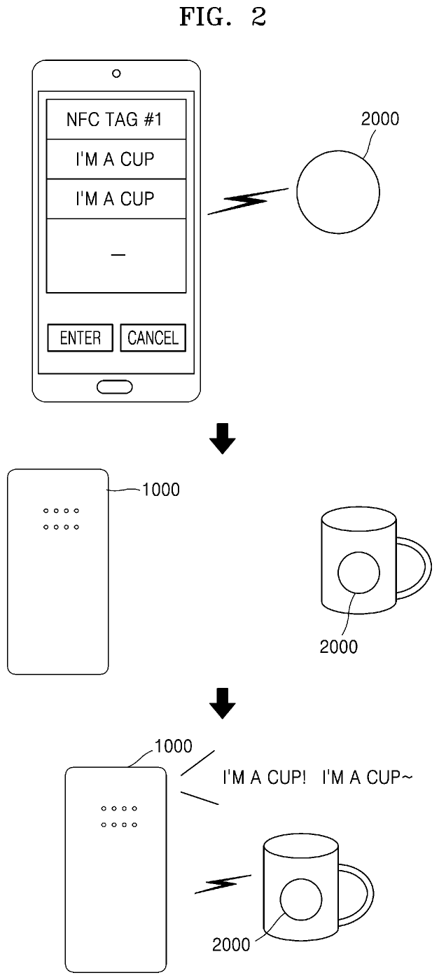

FIG. 1 schematically shows the overall configuration of a system to assist cognitive ability development for the visually impaired or children according to one embodiment of the present invention. FIG. 2 is a diagram for explaining a method of using the cognitive ability development assistance system according to one embodiment of the present invention.

The smartphone terminal of FIG. 1 may correspond to the above-described user terminal, and the NFC tag module may include an antenna 2110 for near field communication (NFC), and an IC chip 2120 including at least one processor and at least one memory. The smartphone terminal may communicate with the NFC tag module 2000 to input or set a specific function to the NFC tag module 2000. According to the present invention, the user may input text information by using a dedicated application of the smartphone terminal, and may attach the NFC tag module 2000 having the inputted text information to an object such as a teaching aid, a toy or a book to use the NFC tag module.

The near field communication (NFC) refers to a proximity wireless communication technology that can communicate data in both directions in a short distance, and the data may be exchanged between terminals in a non-contact manner. NFC is a technology implemented by applying a mobile radio frequency identification (RFID) which is a type of RFID. The RFID may embed a small RFID reader in a smart terminal to provide the user with information about objects to which an RFID tags are attached. Whereas the RFID reader can only read the information of the tag, the NFC has the advantage of being able to read and write information of the tag, and thus the NFC enables data communication between devices. In the present invention, the NFC tag module 2000 configured to be readable and writable as described above, so that the user may input or set a specific function to the NFC tag module 2000 through a smartphone terminal of the user, and may change the set function so as to activate a new function when the function is required to be changed after using the tag. Preferably, the text information inputted by the user using a dedicated application of a smartphone terminal of the user is recorded in the IC chip 2120 of the NFC tag module 2000.

Meanwhile, the auxiliary engineering appliance 1000 may communicate with the NFC tag module 2000 in an NFC communication manner. The NFC tag module 2000 may further store unique identification information, and the auxiliary engineering appliance 1000 may receive the identification information through NFC communication and the text information recorded in the corresponding NFC tag module 2000 through the smart phone terminal, convert the text information into voice information, and output the converted voice information.

Meanwhile, as shown in FIG. 2, text information to be inputted by the user such as “I'm a Cup” may be recorded in the NFC tag module 2000 by using the smartphone terminal of the user.

The NFC tag module 2000 includes an antenna 2110 and an IC chip 2120 and the user of the smartphone terminal, such as a guardian or educator, may attach the NFC tag module 2000 having the inputted text information to a toy, a cup or the like. Thereafter, when a child carrying the auxiliary engineering appliance 1000 tags the auxiliary engineering appliance 1000 on a cup to which the NFC tag module 2000 is attached, the auxiliary engineering appliance 1000 may output the text information “I'm a Cup”, which is inputted using the smartphone terminal, by using a voice through the speaker 1200.

Thus, according to the present invention, the text information is directly inputted into the NFC tag module 2000 and the NFC tag module is attached to an object or book so as to voice-support the text information through the auxiliary engineering appliance 1000, thereby assisting the cognitive ability development of the visually impaired person, visually impaired children, and non-impaired children. For the teaching aids or books of assisting cognitive ability development in the related art, teaching aids required for the above teaching aids, appliances capable of supporting voices, and download contents and books required for the voice support have been necessarily and separately purchased. According to the present invention, only the NFC tag module 2000 and the auxiliary engineering appliance 1000 are used without purchasing all the additional teaching aids and books so that the text information can be conveniently recorded in the NFC tag module 2000 with respect to the existing teaching aids and books used by the parents and child, thereby providing the system of assisting the cognitive ability development so that the user convenience can be cost-effectively improved.

Hereinafter, the auxiliary engineering appliance 1000 and the NFC tag module 2000 will be described in more detail.

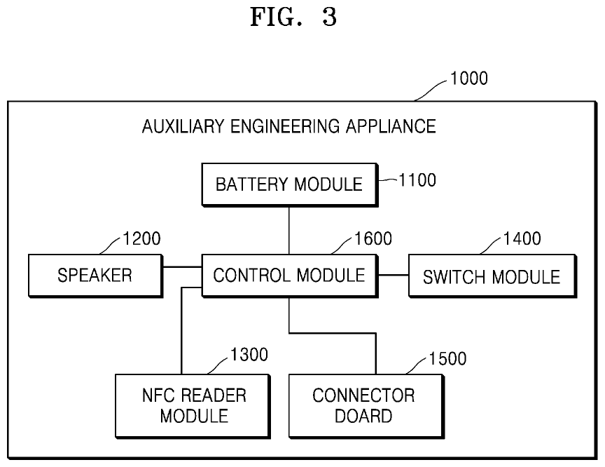

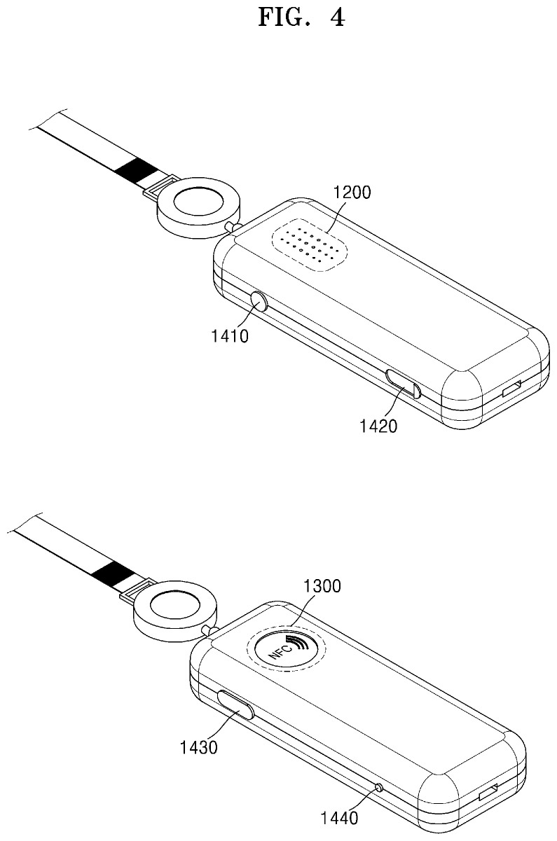

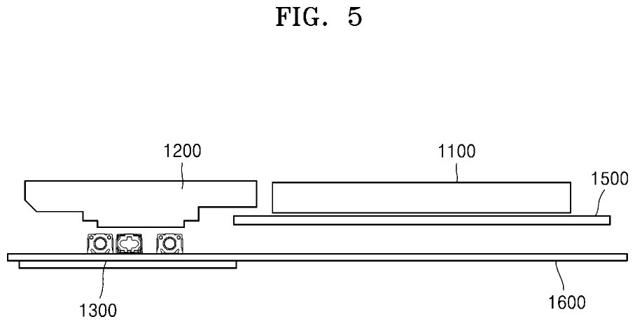

FIG. 3 schematically shows the internal configuration of the auxiliary engineering appliance 1000 according to one embodiment of the present invention. FIGS. 4 and 5 schematically show a side view, a perspective view, and a bottom view in which the internal components of the auxiliary engineering appliance 1000 according to one embodiment of the present invention are combined.

As shown in FIG. 3, the auxiliary engineering appliance 1000 of the present invention includes: a battery module 1100, an NFC reader module 1300, a control module 1600, a speaker 1200, a connector board 1500, and a board support unit 1900.

Specifically, the battery module 1100 supplies power required for the auxiliary engineering appliance 1000.

When tagged on the NFC tag module 2000 in which the text information is inputted, the NFC reader module 1300 may receive the text information recorded in the NFC tag module 2000 in an NFC communication manner.

The control module 1600 may include at least one processor and at least one memory to convert the text information received from the NFC reader module 1300 into voice information.

The speaker 1200 outputs the voice information converted by the control module 1600.

The connector board 1500 connects the control module 1600 to the NFC reader module 1300. Preferably, the NFC reader module 1300 is disposed on one side of the connector board 1500, and the control module 1600, the speaker 1200, and the battery module 1100 are disposed on the other side of the connector board 1500.

As shown in FIG. 4, the battery module 1100 may be disposed on an upper surface of the control module 1600, and the speaker 1200 may be disposed on a side surface of the battery module 1100 in connection with the control module 1600.

In addition, the NFC reader module 1300 may be disposed on a lower surface of the connector board 1500. Accordingly, the NFC reader module 1300 may be separately disposed on one side of the connector board 1500, differently from the control module 1600, the speaker 1200, and the battery module 1100, to transmit and receive NFC communication signals more smoothly when an external NFC tag module 2000 is tagged, thereby increasing the recognition rate of the NFC tag module 2000, so that the user convenience can be improved.

In addition, as shown in FIG. 5(a), the auxiliary engineering appliance 1000 may include a board support unit 1900 disposed on an upper surface of the connector board 1500 to support the control module 1600 from the connector module while being spaced apart from the connector module by a predetermined interval, and one or more auxiliary engineering appliance 1000 components including a USB connector unit for charging the switch module 1400 and the battery module 1100 may be disposed in a space between the control module 1600 and the connector board 1500 spaced apart by the predetermined interval. Because mainly used by the visually impaired or non-impaired children, the auxiliary engineering appliance 1000 of the present invention is required to be prevented from being easily broken by impacts caused when, for example, the children throw the auxiliary engineering appliance 1000. The present invention may provide the auxiliary engineering appliance 1000 protected from external impacts to have the enhanced durability through the structure of the above-mentioned components. In addition, as shown in FIG. 5(b), the auxiliary engineering appliance 1000 having the improved convenience may be provided, in which the NFC reader module 1300 may be disposed on the other side disposed thereon with the control module 1600, the speaker 1200, and the battery module 1100, to enable the communication signals to be transmitted and received more smoothly when the NFC tag module 2000 is tagged, so as to be used by the visually impaired infants and toddlers, the severe or multiple visually impaired people, or the non-impaired children more easily.

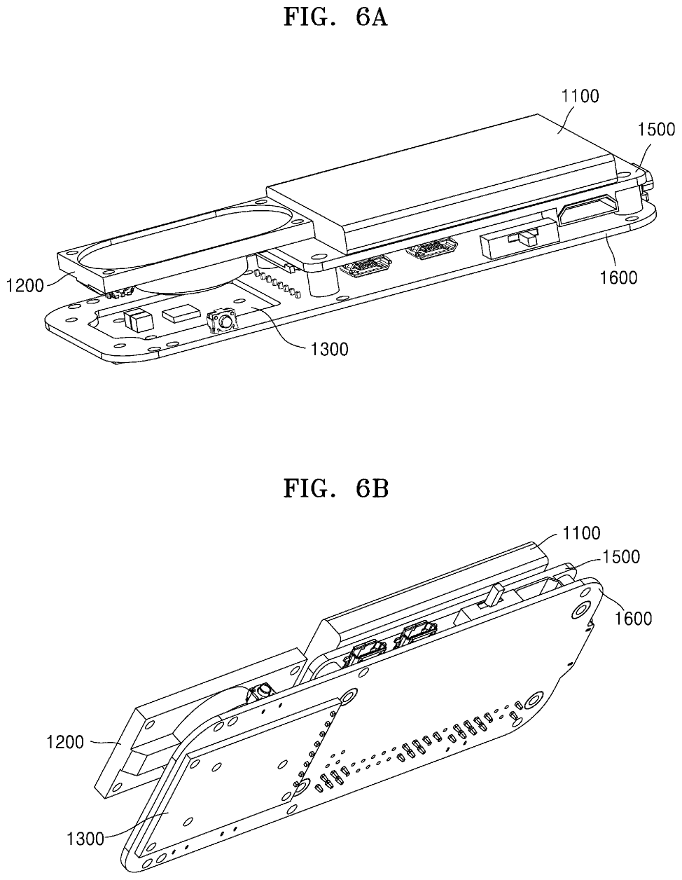

FIGS. 6A and 6B schematically shows the configuration of the auxiliary engineering appliance 1000 according to one embodiment of the present invention.

The auxiliary engineering appliance 1000 of the present invention may further include an upper case 1700, a lower case 1800, and a switch module 1400, wherein the battery module 1100, the NFC reader module 1300, the control module 1600, the speaker 1200, the connector board 1500, the board support unit 1900 and the switch module 1400 may be disposed in a space formed by the upper case 1700 and the lower case 1800. The upper case 1700 and the lower case 1800 may be applied as a shock-absorbing bumper case that may be protected from external impacts. In addition, the upper case 1700 may include a plurality of speaker holes 1710 through which the voice outputted from the speaker 1200 is exposed. An NFC reader indication unit 1810, which indicates that the NFC reader module 1300 is accommodated in the inner space of the lower case 1800, may be formed in the lower case 1800. Accordingly, a speaker hole 1710 for outputting the voice from the speaker 1200 is configured in the upper case 1700, and the NFC reader indication unit 1810 indicating that the NFC reader module 1300 is present on the surface of the lower case 1800 accommodated in the inner space of the NFC reader module 1300 is configured, thereby enabling children to more easily distinguish a front side and a rear side of the auxiliary engineering appliance 1000, so as to easily tag the auxiliary engineering appliance 1000 to the NFC tag module 2000.

Meanwhile, as shown in FIGS. 6A and 6B, the switch module 1400 may be disposed in the space between the control module 1600 and the connector module spaced apart by the predetermined interval caused by the board support unit 1900, and the switch module 1400 includes: a power switch 1410 capable of controlling On/Off of the auxiliary engineering appliance 1000; an NFC tagging switch 1420 capable of activating tagging of the NFC reader module 1300 and the NFC tag module 2000; a Bluetooth pairing switch 1430 capable of performing Bluetooth pairing with a smartphone terminal; and a voice control switch 1440 capable of controlling speed and volume of a voice outputted to the speaker 1200.

Specifically, the power switch 1410 may be operated to control an Off-mode and an On-mode, and the On-mode may include a Bluetooth low energy (BLE) mode and a standlone mode, in which Bluetooth communication may be performed with the smartphone terminal upon operation in the BLE mode, and the tagging only through NFC communication may be performed upon operation in the standalone mode.

According to the NFC tagging switch 1420, when the interval between the NFC reader module 1300 and the NFC tag module 2000 is within a predetermined distance, the tagging is activated according to the input of the NFC tagging switch 1420 and the NFC communication is performed, so that the NFC reader module 1300 may receive identification information and text information of the NFC tag module 2000.

The Bluetooth pairing switch 1430 may enable Bluetooth pairing with the smartphone terminal to register device information about the auxiliary engineering appliance 1000 of the smartphone terminal of the user such as a guardian and educator, and may be set to be connected automatically after connected once.

The voice control switch 1440 may be configured to adjust the speed and volume of the voice outputted to the speaker 1200. When the text information registered in the NFC tag module 2000 is outputted too fast via a voice in a viewpoint of the level of a child using the auxiliary engineering appliance 1000, the speed may be adjusted to be slow by according to the user's input. When the voice is outputted too slow, the speed may be adjusted to be faster by according to the user's input. In addition, when the volume of the voice is too small or too loud, the volume may be adjusted according to the user's input. For example, the volume control may be performed when the voice control switch 1440 is pressed short, and the speed control may be performed when the voice control switch is pressed long.

Preferably, the switches of the auxiliary engineering appliance 1000 may be formed in coupling portions of the upper case 1700 and the lower case 1800 in different shapes, respectively, such that the visually impaired people can distinguish each switch more easily.

In addition, the auxiliary engineering appliance 1000 may further include an LED module for informing a capacity status of a battery so that the capacity of the battery may be indicated in various colors according to the battery capacity status. In addition, a necklace-shaped wheel may be configured for the convenience of the child using the auxiliary engineering appliance 1000 so as to be easily tagged on the object or book to which tag module 2000 is attached, while elongating or contracting the length of the auxiliary engineering appliance 1000.

In the above configuration, according to the auxiliary engineering appliance 1000 of the present invention, the visual impaired child as the main user or the non-impaired infant or child is allowed to perform the input to adjust the speed of a supported voice so as to perform self-motivated learning based on the learning ability of the child, and even the visually impaired child can easily and tactilely distinguish the shape of the auxiliary engineering appliance, so that the user convenience can be improved.

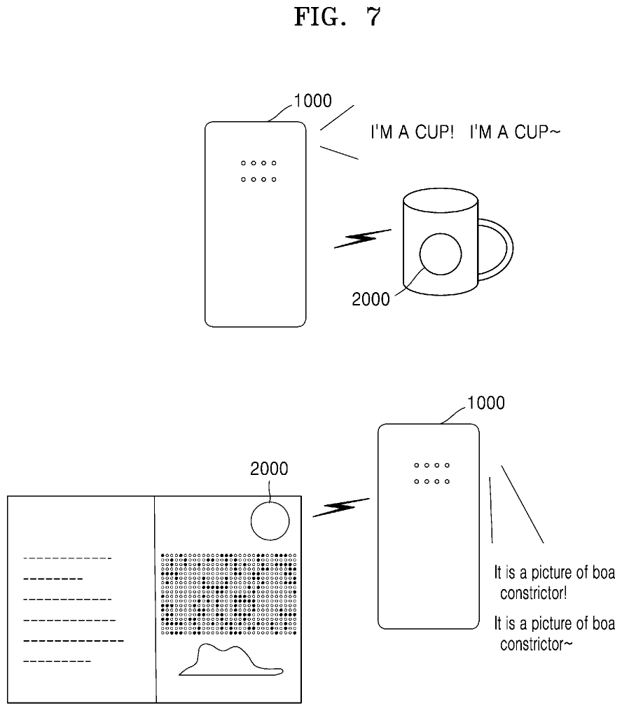

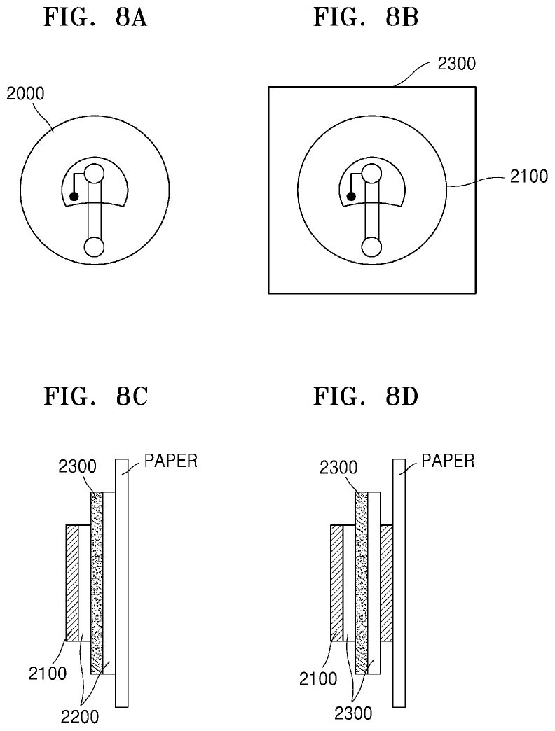

FIG. 7 is a view explaining an example of applying the system of assisting the cognitive ability development according to one embodiment of the present invention onto a book. FIGS. 8A, 8B, 8C and 8D schematically show the NFC tag module 2000 according to one embodiment of the present invention.

As shown in FIG. 7, the NFC tag module 2000 may be attached to objects such as teaching aids and toys as described above, and voice information such as “I am a cup” may be inputted therein, or the NFC tag module may be attached to one side of a book so that corresponding contents, such as “It was a picture of a boa constrictor!”, of the book may be inputted and outputted via a voice. Meanwhile, FIG. 8A schematically shows an object NFC tag module 2000 that may be attached to objects such as teaching aids and toys, and FIG. 8B schematically shows a book NFC tag module 2000 that may be attached to a book.

Referring to FIGS. 8A and 8B, the book NFC tag module 2000 may further include an electromagnetic wave blocking layer 2300 having an area larger than the NFC tag. In general, since one NFC tag module 2000 is attached to and used for an object such as a teaching aid and a toy, a signal interference problem due to the use of multiple tags rarely occurs. However, when the NFC tag module 2000 is used to read the contents of the book, a plurality of NFC tag modules 2000 are overlapped to easily cause the signal interference. In order to solve the above problem, the NFC tag module 2000 of the present invention may include the electromagnetic wave blocking layer 2300, so that the above signal interference problem may be solved.

FIG. 8C shows an example of the NFC tag module 2000 that may be used to be attached to one side of a book. As shown in FIG. 8C, the NFC tag module 2000 includes: a first NFC tag 2100 including an antenna 2110 and an IC chip 2120; a first adhesive layer 2200.1 disposed on the first NFC tag 2100 and having a sectional area corresponding to the first NFC tag 2100; an electromagnetic wave blocking layer 2300 disposed on the first adhesive layer 2200.1 and having an area greater than the first NFC tag 2100; and a second adhesive layer 2200.2 disposed on the electromagnetic wave blocking layer 2300 and having a sectional area corresponding to the electromagnetic wave blocking layer 2300.

According to the NFC tag module 2000, an NFC tag including an IC chip 2120 recording text information inputted by a user through a smartphone terminal may be attached to the electromagnetic wave blocking layer 2300 having an area greater than the NFC tag, and attached to one side of the book, so that the problem caused by the signal interference may be solved. In addition, the NFC tag module 2000 formed by combining the first NFC tag 2100, the first adhesive layer 2200.1, the electromagnetic wave blocking layer 2300 and the second adhesive layer 2200.2 with each other may be configured to have a thin form to be prevented from being thicker than the height of the braille, so that the book can be prevented from being thick even when the NFC tag module is attached to a braille book.

Meanwhile, FIG. 8D shows an example of an NFC tag module 2000 attached and used after the contents of both sides of a book are inputted to one NFC tag module 2000. As shown in FIG. 8D, the NFC tag module 2000 may be attached to one side of the book, the NFC tag module 2000 includes: a first NFC tag 2100.1 including an antenna 2110 and an IC chip 2120; a first adhesive layer 2200.1 disposed on the first NFC tag 2100.1 and having a sectional area corresponding to the first NFC tag 2100.1; an electromagnetic wave blocking layer 2300 disposed on the first adhesive layer 2200.1 and having an area greater than the first NFC tag 2100.1; a second adhesive layer 2200.2 disposed on the electromagnetic wave blocking layer 2300 and having a sectional area corresponding to the electromagnetic wave blocking layer 2300; and a second NFC tag 2100.2 disposed on the second adhesive layer 2200.2 and having an area smaller than the second adhesive layer 2200.2.

According to the NFC tag module 2000, an NFC tag including an IC chip 2120 recording text information inputted by a user through a smartphone terminal may be attached to the electromagnetic wave blocking layer 2300 having an area greater than the NFC tag, and the electromagnetic wave blocking layer 2300 and a second NFC tag 2100.2 having an area smaller than the electromagnetic wave blocking layer 2300 may be disposed on sides of the second adhesive layer 2200.2 having a sectional area corresponding to the electromagnetic wave blocking layer 2300, respectively, so that the NFC tag module 2000 may be attached to one side of the book in a manner that the second adhesive layers 2200.2 to which the second NFC tag 2100.2 is not attached is partially attached to paper. According to the NFC tag module 2000 having the above configuration, even when the NFC tag module 2000 is attached to only one side of the book, the contents of one side of the book may be inputted to the first NFC tag 2100.1, and the contents of the other side of the book may be inputted the second NFC tag 2100.2, so that all the contents of the both sides of the book may be inputted, and the problem caused by the signal interference may also be solved by the electromagnetic wave blocking layer 2300 having the area greater than each of the NFC tags.

In addition, the above NFC tag module 2000 of the present invention is not limited to the Braille book, also may be applied to books that general non-impaired children can read, and may be applied to both objects and books to which the NFC tag module 2000 can be attached, such as teaching aids for a braille play using magnets.

In the above manners, the system of assisting cognitive ability development of the present invention may use the both-sided NFC tag module 2000 so that text information can be inputted to read the contents of both sides of the book even when the NFC module is attached to only one side of the book, and thus the interference of NFC signals upon attachment of multiple NFC tag modules 2000 can be prevented.

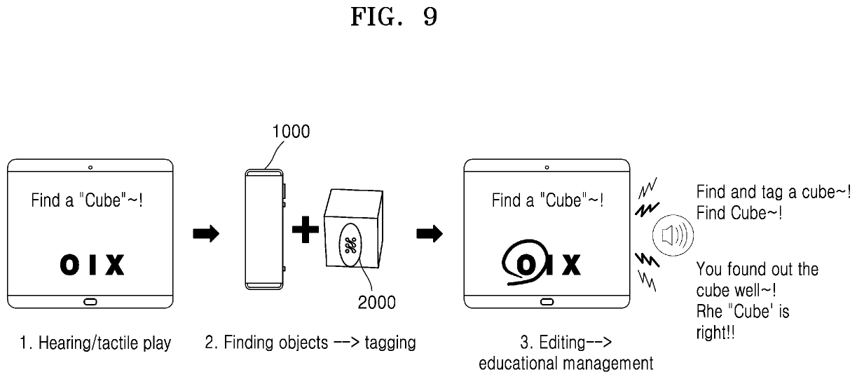

FIG. 9 is a diagram for explaining a method of using the cognitive ability development assistance system according to one embodiment of the present invention.

The system of assisting cognitive ability development of the present invention may be linked with a smartphone terminal, and the smartphone terminal may be provided with an educational application for children. As shown in FIG. 9, questions related to learning, such as “Find the cube!” may be provided through a voice, a child hearing the question may find a cube and tag the cube on the auxiliary engineering appliance 1000 to determine whether the presented question is correct or not, and a voice for the correct answer may be provided. Thus, according to the present invention, the children using the system of assisting the cognitive ability development are allowed to learn about the combination of the teaching aids, so that various learning methods can be provided.

One embodiment of the present invention may be implemented in the form of a recording medium containing instructions executable by a computer, such as a program module executed by the computer. The computer-readable medium may be any available medium accessible by the computer, and include a volatile or non-volatile medium, and a removable or non-removable medium. In addition, the computer-readable medium may include a computer storage medium and a communication medium. The computer storage medium includes all of volatile, non-volatile, removable and non-removable media implemented by any method or technology for storing information such as computer readable instructions, data structures, program modules or other data. The communication medium typically includes computer readable instructions, data structures, program modules, or other data of a modulated data signal such as a carrier wave, or other transmission mechanism, and includes any information delivery medium.

Although the method and system of the present invention have been described in connection with specific embodiments, some or all of components or operations thereof may be implemented using a computer system having a general hardware architecture.

The above description of the present invention is merely for illustration, and it will be apparent that a person having ordinary skill in the art may carry out various deformations and modifications within the scope without departing from the idea of the present invention, the following claims and equivalents thereof. Therefore, the above described embodiments should be understood in all respects as illustrative and not restrictive. For example, each component described as unitary may be implemented in a distributed manner. Likewise, components that are described as distributed may also be implemented in a combined form.

The scope of the invention is indicated by the following claims rather than the above detailed description, and all deformations or modifications derived from the idea and scope of the claims and their equivalents should be construed as being included in the scope of the present invention.

What is claimed is:

1. A system of assisting cognitive ability development for a visually impaired or children, the system comprising:
an NFC tag module including an antenna and an IC chip and configured to be readable and writable; and
an auxiliary engineering appliance, wherein
the auxiliary engineering appliance includes:
a battery module;
an NFC reader module for receiving text information recorded in the NFC tag module by using an NFC communication scheme;
a control module for converting the text information received from the NFC reader module into voice information;
a speaker for outputting the voice information converted by the control module; and
a connector board for connecting the control module to the NFC reader module.

2. The system of claim 1, wherein the NFC tag module includes an IC chip in which text information inputted by a user using a dedicated application on a smartphone terminal of the user is recorded.

3. The system of claim 1, wherein the NFC reader module is disposed on one side of the connector board, and the control module, the speaker, and the battery module are disposed on an opposite side of the connector board.

4. The system of claim 1, wherein the auxiliary engineering appliance further includes a switch module including:
a power switch capable of controlling On/Off of the auxiliary engineering appliance;
an NFC tagging switch capable of activating tagging of the NFC reader module and the NFC tag module;
a Bluetooth pairing switch capable of performing Bluetooth pairing with a smartphone terminal; and
a voice control switch capable of controlling speed and volume of a voice outputted to the speaker.

5. The system of claim 1, wherein the NFC tag module is attachable to one side of a book, and the NFC tag module includes:
a first NFC tag including an antenna and an IC chip;
a first adhesive layer disposed on the first NFC tag and having a sectional area corresponding to the first NFC tag;
an electromagnetic wave blocking layer disposed on the first adhesive layer and having an area greater than the first NFC tag;
a second adhesive layer disposed on the electromagnetic wave blocking layer and having a sectional area corresponding to the electromagnetic wave blocking layer; and
a second NFC tag disposed on the second adhesive layer and having an area smaller than the second adhesive layer.

6. A method of assisting cognitive ability development by using a system of assisting cognitive ability development for a visually impaired or children, wherein the system comprises:
an NFC tag module including an antenna and an IC chip and configured to be readable and writable; and
an auxiliary engineering appliance, wherein
the auxiliary engineering appliance includes:
a battery module;
an NFC reader module for receiving text information recorded in the NFC tag module by using an NFC communication scheme;
a control module for converting the text information received from the NFC reader module into voice information;
a speaker for outputting the voice information converted by the control module; and
a connector board for connecting the control module to the speaker and the NFC reader module, and wherein the method comprises:
inputting, by a user, text information by using a dedicated application on a smartphone terminal of the user, and
recording the inputted text information in the NFC tag module by using NFC communication between the NFC tag module and the smart phone terminal;
receiving, by the auxiliary engineering appliance, the text information recorded in the NFC tag module by using an NFC communication scheme;
converting, by the auxiliary engineering appliance, the text information received from the NFC reader module into voice information; and
outputting, by the auxiliary engineering appliance, the voice information converted by the control module.
